(19) United States
(12) Patent Application Publication
(10) Pub. No.: US 2026/0335596 A1
(43) Pub. Date: Oct. 29, 2026

(54) PUNCTURE CATHETER

(71) Applicant: KANEKA CORPORATION, Osaka (JP)

(72) Inventors: Hirokazu Kamakura, Nagano (JP); Yuki Mukai, Nagano (JP)

(73) Assignee: KANEKA CORPORATION, Osaka (JP)

(21) Appl. No.: 19/677,718

(22) Filed: May 14, 2026

Related U.S. Application Data
(63) Continuation of application No. PCT/JP2024/041410, filed on Nov. 22, 2024.

(30) Foreign Application Priority Data
Dec. 21, 2023 (JP) 2023-215765

(51) Int. Cl. A61M 25/00; A61M 25/01

(52) U.S. Cl. CPC A61M 25/0084; A61M 25/0136; A61M 2025/0089; A61M 2205/3303

(57) ABSTRACT

A puncture catheter includes a resin tube that has a longitudinal direction and that has a lumen extending in the longitudinal direction, a tubular member that has an outer surface in contact with an inner surface of a distal portion of the resin tube, and a puncture needle that is for medical use and that is movable in the lumen of the resin tube and the lumen of the tubular member in the longitudinal direction of the resin tube. In the puncture catheter, the tubular member is constituted by a braid in which a wire is braided, a coil around which a wire is wound, a pipe in which a plurality of holes or grooves are formed, or a combination thereof.

### TECHNICAL FIELD

One or more embodiments of the present invention relate to a puncture catheter including a puncture needle for medical use.

### BACKGROUND

A drug solution such as a cardiac regenerative cell preparation is directly administered to cardiac muscle cells which are about to lose their functions due to myocardial infarction or the like, thereby performing treatment or the like for regenerating the cardiac muscle cells. When a drug solution is directly administered as described above to an organ in a body, it is necessary to insert a catheter including a needle into a cavity of the body and to puncture the organ with the needle.

As a system for use in the above-described treatment, PTL 1 discloses a drug-solution injection needle system including a hollow needle for puncturing the myocardium of a patient and injecting a drug solution and a guiding catheter into which the needle is inserted.

### PATENT LITERATURE

PTL 1: International Publication No. WO2021/192283

However, in the drug-solution injection needle system described in PTL 1, the needle easily sticks into a wall of the guiding catheter and the needle easily passes through the wall of the guiding catheter.

### SUMMARY

One or more embodiments of the present invention have been made in view of the above circumstances, and to provide a novel puncture catheter in which a puncture needle is less likely to stick into a resin tube and the puncture needle is less likely to pass through the resin tube.

A puncture catheter according to one or more embodiments of the present invention, in which the above have been addressed, is as follows.
[1] A puncture catheter including:
a resin tube that has a longitudinal direction and that has a lumen extending in the longitudinal direction;
a tubular member that has an outer surface in contact with an inner surface of a distal portion of the resin tube; and
a puncture needle that is for medical use and that is movable in the longitudinal direction in the lumen of the resin tube and a lumen of the tubular member,
in which the tubular member is formed of a braid in which a wire is braided, a coil around which a wire is wound, a pipe in which a plurality of holes or grooves are formed, or a combination thereof.

In the puncture catheter, since the outer surface of the tubular member is in contact with the inner surface of the resin tube, the puncture needle is less likely to stick into the resin tube and the puncture needle is less likely to pass through the resin tube.

The puncture catheter according to one or more embodiments of the present invention may be any one of the following [2] to [7].
[2] The puncture catheter according to [1],
in which a gap is present in the lumen of the tubular member, and
in which an inner surface of the tubular member is exposed to the gap.
[3] The puncture catheter according to [1] or [2],
in which the puncture catheter includes a handle that controls a curving direction of the resin tube,
in which the resin tube has a curving section that is curved by operating the handle and a non-curving section that is located proximal to the curving section and that is not curved by operating the handle, and
in which the tubular member is disposed in the curving section.
[4] The puncture catheter according to [3], in which the tubular member is not disposed in the non-curving section.
[5] The puncture catheter according to any one of [1] to [4],
in which three or more electrodes are disposed on an outer surface of the resin tube, and
in which the tubular member is disposed distal to a proximal end of an electrode of the three or more electrodes located most proximally and is not disposed proximal to a proximal end of the electrode located most proximally.
[6] The puncture catheter according to any one of [1] to [5], in which the tubular member is made of a metal.
[7] The puncture catheter according to any one of [1] to [6], in which an inner surface of the tubular member and the puncture needle are contactable with each other.

### Advantageous Effects of Invention

In the puncture catheter according to one or more embodiments of the present invention, since the outer surface of the tubular member is in contact with the inner surface of the resin tube, it is difficult for the puncture needle to stick into the resin tube and to pass through the resin tube.

### BRIEF DESCRIPTION OF THE DRAWINGS

FIG. 1 is a side view showing an example of a puncture catheter according to one or more embodiments of the present invention.

FIG. 2 is a sectional view (partial side view) of the puncture catheter shown in FIG. 1.

FIG. 3 is a sectional view (partial side view) showing a state where the puncture catheter shown in FIG. 2 is curved.

FIG. 4 is a sectional end view taken along the line IV-IV shown in FIG. 2.

FIG. 5 is a sectional view (partial side view) showing a modification of the puncture catheter shown in FIG. 2.

FIG. 6 is a sectional view (partial side view) showing a modification of the puncture catheter shown in FIG. 2.

FIG. 7 is a sectional view (partial side view) showing a modification of the puncture catheter shown in FIG. 2.

FIG. 8 is a sectional view showing a modification of a tubular member.

### DETAILED DESCRIPTION

Hereinafter, one or more embodiments of the present invention will be described in detail with reference to the drawings. However, the present invention is not limited to the examples shown in the drawings and can be carried out with changes added thereto, as appropriate, within a range conforming to the gist described above and below, and these changes are also included in the technical scope of one or more embodiments of the present invention. In the drawings, hatching, reference signs, and the like are sometimes omitted for convenience. In such a case, the description and other drawings are referred to. Also, the dimensions of the various components in the drawings may differ from the actual dimensions in order to facilitate understanding of the features of one or more embodiments of the present invention.

A puncture catheter according to one or more embodiments of the present invention is a puncture catheter including a resin tube that has a longitudinal direction and that has a lumen extending in the longitudinal direction, a tubular member that has an outer surface in contact with an inner surface of a distal portion of the resin tube, and a puncture needle that is for medical use and that is movable in a lumen of the resin tube and a lumen of the tubular member in the longitudinal direction of the resin tube, the puncture catheter being characterized in that the tubular member is constituted by a braid in which a wire is braided, a coil around which a wire is wound, a pipe in which a plurality of holes or grooves are formed, or a combination thereof.

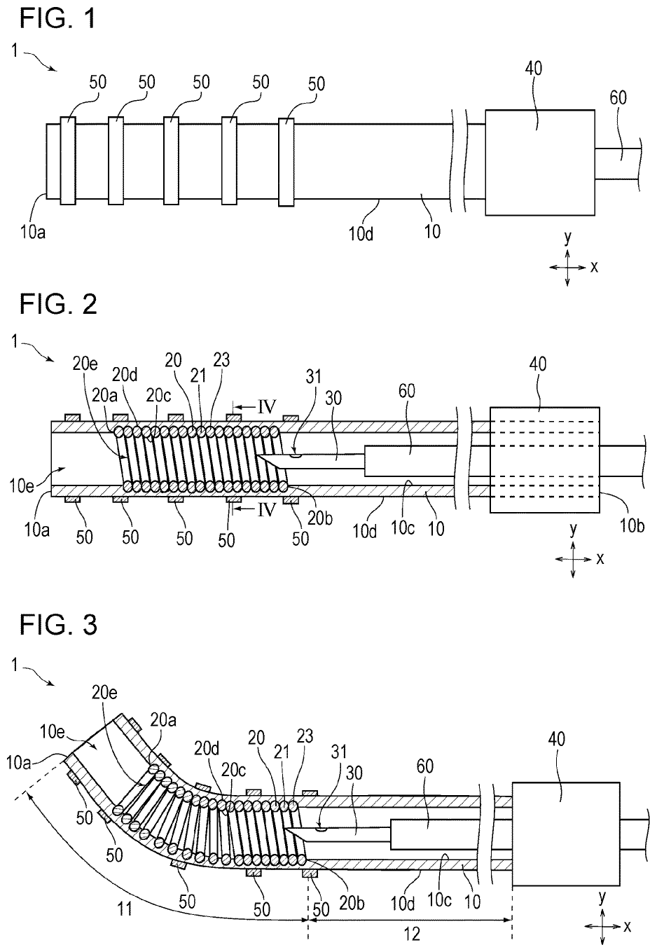

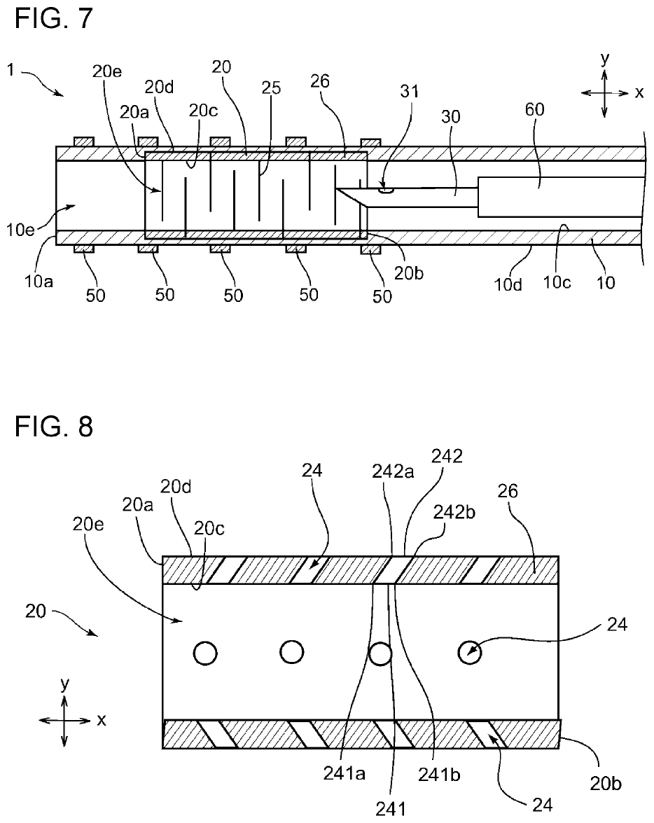

An overall configuration of a puncture catheter according to one or more embodiments of the present invention will be described with reference to FIGS. 1 to 8. FIGS. 1 to 7 show a puncture catheter 1 including a resin tube 10, a tubular member 20, and a puncture needle 30.

FIGS. 2 to 8 show specific forms of the tubular member 20.

In the drawings, the longitudinal direction of the resin tube 10 is indicated by x, the radial direction of the resin tube 10 is indicated by y, and the circumferential direction of the resin tube 10 is indicated by c. The radial direction y is a direction perpendicular to the longitudinal direction x. In other words, the longitudinal direction X of the resin tube 10 can be said to be an extending direction of the resin tube 10.

In the present description, the proximal side refers to a direction on the hand side of a user in the longitudinal direction X of the resin tube 10, and the distal side is opposite to the proximal side, that is, a direction on the side of a treatment target. In addition, when each member or each portion is divided into two equal parts in the longitudinal direction x of the resin tube 10, a distally located part of each member or each portion is referred to as a distal portion of each member or each portion, and a proximally located part of each member or each portion is referred to as a proximal portion of each member or each portion. The distal end of each member or each portion is a most distally located end of each member or each portion. The proximal end of each member or each portion is a most proximally located end of each member or each portion. An end portion includes a portion around an end. That is, a distal end portion refers to a distal end and a portion around the distal end, and a proximal end portion refers to a proximal end and a portion around the proximal end.

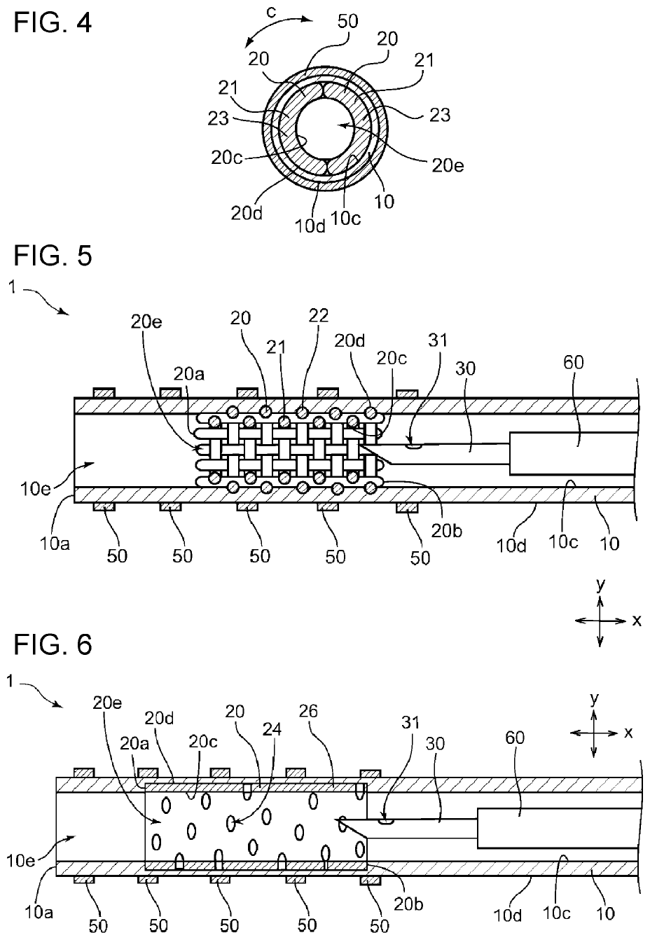

FIG. 1 is a side view showing an example of a puncture catheter according to one or more embodiments of the present invention. FIG. 2 is a sectional view (partial side view) of the puncture catheter shown in FIG. 1. FIG. 3 is a sectional view (partial side view) showing a state where the puncture catheter shown in FIG. 2 is curved. FIG. 4 is a sectional end view taken along the line IV-IV shown in FIG. 2. FIGS. 5 to 7 are sectional views (partial side views) showing modifications of the puncture catheter shown in FIG. 2. FIG. 8 is a sectional view showing a modification of a tubular member.

As shown in FIGS. 1 to 7, the puncture catheter 1 includes the resin tube 10, the tubular member 20, and the puncture needle 30.

As shown in FIGS. 1 and 2, the resin tube 10 has the longitudinal direction x and has a lumen 10e extending in the longitudinal direction x. The resin tube 10 has a distal end 10a, a proximal end 10b, an inner surface 10c facing the lumen 10e, and an outer surface 10d facing the outside of the resin tube 10. The distal end 10a and the proximal end 10b of the resin tube 10 may be open.

As shown in FIGS. 2 to 8, the tubular member 20 is a member having a tubular shape and has a distal end 20a, a proximal end 20b, a lumen 20e, an inner surface 20c facing the lumen 20e, and an outer surface 20d facing the outside of the tubular member 20. The lumen 20e of the tubular member 20 extends in the longitudinal direction x of the resin tube 10, and the distal end 20a and the proximal end 20b may be open.

As shown in FIGS. 2 to 7, the outer surface 20d of the tubular member 20 is in contact with the inner surface 10c of a distal portion of the resin tube 10. Only a portion of the outer surface 20d of the tubular member 20 may be in contact with the inner surface 10c of the distal portion of the resin tube 10, or the entirety of the outer surface 20d of the tubular member 20 may be in contact with the inner surface 10c of the distal portion of the resin tube 10.

The tubular member 20 is constituted by a braid 22, such as that shown in FIG. 5, in which a wire 21 is braided, a coil 23, such as that shown in FIG. 2, around which the wire 21 is wound, a pipe 26, such as that shown in FIGS. 6 to 8, in which a plurality of holes 24 or grooves 25 are formed, or a combination thereof.

The puncture needle 30 is for medical use and is configured to puncture a target tissue. The puncture needle 30 is movable in the longitudinal direction x of the resin tube 10 in the lumen 10e of the resin tube 10 and the lumen 20e of the tubular member 20.

In the above-described puncture catheter 1, since the outer surface 20d of the tubular member 20 is in contact with the inner surface 10c of the resin tube 10, the puncture needle 30 is less likely to stick into the resin tube 10 and the puncture needle 30 is less likely to pass through the resin tube 10.

The puncture catheter 1 is to be used to administer a liquid such as a cell preparation or a drug solution to a target tissue. Specifically, the puncture catheter 1 can be used for direct administration to an organ in a body, for example, a heart, a kidney, a liver, or the like. For example, the puncture catheter 1 can be used to directly administer an iPS cell suspension to the liver or the kidney or to directly administer a cardiac regenerative cell preparation to the heart, more specifically, the cardiac muscle. Note that an organ in the body refers to an organ in the body, particularly, in the abdomen or chest and is also referred to as an internal organ.

The resin tube 10 may have a tubular shape, a hollow columnar shape, a hollow polygonal columnar shape, or a shape obtained by combining these shapes.

The resin tube 10 can be manufactured by extrusion molding. The resin tube 10 may be formed of a single layer or a plurality of layers. A portion of the resin tube 10 in the longitudinal direction x or the circumferential direction c may be formed of a single layer while the other portion may be formed of a plurality of layers.

The resin tube 10 may be made of, for example, a synthetic resin such as a polyolefin resin (for example, polyethylene or polypropylene), a polyamide resin (for example, nylon), a polyester resin (for example, PET), an aromatic polyether ketone resin (for example, PEEK), a polyether polyamide resin, a polyurethane resin, a polyimide resin, or a fluororesin (for example, PTFE, PFA, or ETFE). These may be used alone or in combination of two or more kinds thereof.

The length of the resin tube 10 can be 900 mm or longer, 1000 mm or longer, 1100 mm or longer, or the like. The length of the resin tube 10 can be 1200 mm or less, 1400 mm or less, 1600 mm or less, or the like. The length of the resin tube 10 refers to the longest length among lengths of the resin tube 10 in the longitudinal direction x of the resin tube 10. For use as a puncture catheter for puncturing the myocardium, the length of the resin tube 10 may be 1400 mm.

The outer diameter of the resin tube 10 can be 0.5 mm or more, 1.0 mm or more, 1.5 mm or more, or the like. The outer diameter of the resin tube 10 can be 2.0 mm or less, 2.5 mm or less, 3.0 mm or less, or the like. In the case where the outer shape of the resin tube 10 as observed in the longitudinal direction x of the resin tube 10 is not a perfect circle, the diameter of a circle circumscribing the resin tube 10 is defined as the outer diameter of the resin tube 10. For use as a puncture catheter for puncturing the myocardium, the outer diameter of the resin tube 10 may be 1 mm.

Although the outer surface 20d of the tubular member 20 is in contact with the inner surface 10c of the distal portion of the resin tube 10, the outer surface 20d of the tubular member 20 may be in contact with the inner surface 10c of the distal end portion of the resin tube 10.

Although only a portion of the outer surface 20d of the tubular member 20 may be in contact with the inner surface 10c of the distal portion of the resin tube 10, the entirety of the outer surface 20d of the tubular member 20 may be in contact with the inner surface 10c of the distal portion of the resin tube 10.

The tubular member 20 may be made of a resin. In this case, although the resin constituting the tubular member 20 may have flexural rigidity higher or lower than that of a resin constituting the resin tube 10, the resin constituting the tubular member 20 may have flexural rigidity lower than that of the resin constituting the resin tube 10. Thus, the puncture catheter 1 is easily curved at a part where the tubular member 20 is disposed. The tubular member 20 may be made of a metal. The tubular member 20 may be entirely made of only a metal or may be entirely made of only a resin. A portion of the tubular member 20 may be made of a metal while the other portion is made of a resin.

The metal constituting the tubular member 20 is, for example, stainless steel such as SUS304 or SUS316, platinum, nickel, cobalt, chromium, titanium, tungsten, gold, a Ni—Ti alloy, a Co—Cr alloy, or a combination thereof.

An example of the resin constituting the tubular member 20 is, for example, polyparaphenylene terephthalamide, polyether ether ketone (PEEK), polycarbonate (PC), or the like.

The outer surface 20d of the tubular member 20 and the inner surface 10c of the distal portion of the resin tube 10 may be fixed to each other. The outer surface 20d of the tubular member 20 and the inner surface 10c of the distal portion of the resin tube 10 may be welded to each other by being partially melted or may be bonded to each other with an adhesive.

As shown in FIGS. 2 to 8, a gap is present in the lumen 20e of the tubular member 20, and the inner surface 20c of the tubular member 20 may be exposed to the gap. Although only a portion of the inner surface 20c of the tubular member 20 may be exposed to the gap, the entirety of the inner surface 20c of the tubular member 20 may be exposed to the gap. With the above configuration, the inner surface 10c of the resin tube 10 can be easily protected by the tubular member 20.

The puncture needle 30 can come into contact with the inner surface 20c of the tubular member 20. With the above-described configuration, the inner surface 10c of the resin tube 10 can be easily protected by the tubular member 20. Note that only a portion of the inner surface 20c of the tubular member 20 may be able to come into contact with the puncture needle 30, or the entirety of the inner surface 20c of the tubular member 20 may be able to come into contact with the puncture needle 30.

No other member may be disposed between the inner surface 20c of the tubular member 20 and the puncture needle 30. For example, another resin layer may be not provided on the inner surface 20c of the tubular member 20.

The maximum outer diameter of the tubular member 20 may be larger than the minimum inner diameter of the resin tube 10. The maximum outer diameter of the tubular member 20 may be the same as the minimum inner diameter of the resin tube 10.

The minimum inner diameter of the tubular member 20 may be smaller than the minimum inner diameter of the resin tube 10. The minimum inner diameter of the tubular member 20 and the minimum inner diameter of the resin tube 10 may be the same. When the minimum inner diameter of the tubular member 20 is the same as the minimum inner diameter of the resin tube 10, it is possible to easily eliminate a step between the resin tube 10 and the tubular member 20. Thus, the inner surface 10c of the resin tube 10 can be easily protected by the tubular member 20 while the puncture needle 30 is suppressed from being caught by the step.

The braid 22 is configured such that the maximum length of the interval between portions of the wire 21 adjacent to each other in the longitudinal direction x of the resin tube 10 may be 2 times or less, 1.8 times or less, or 1.5 times or less of the wire diameter of the wire 21 in a state where no external force is applied to the puncture catheter 1. The braid 22 may be configured such that the maximum length of the interval between portions of the wire 21 adjacent to each other in the longitudinal direction x of the resin tube 10 is less than or equal to the wire diameter of the wire 21 in a state where no external force is applied to the puncture catheter 1. With the above configuration, the inner surface 10c of the resin tube 10 can be easily protected by the tubular member 20.

The braid 22 is configured such that the maximum length of the interval between portions of the wire 21 adjacent to each other in the radial direction y of the resin tube 10 may be 2 times or less, 1.8 times or less, or 1.5 times or less of the wire diameter of the wire 21 in a state where no external force is applied to the puncture catheter 1. The braid 22 may be configured such that the maximum length of the interval between portions of the wire 21 adjacent to each other in the radial direction y of the resin tube 10 is less than or equal to the wire diameter of the wire 21 in a state where no external force is applied to the puncture catheter 1. With the above configuration, the inner surface 10c of the resin tube 10 can be easily protected by the tubular member 20.

In a state where no external force is applied to the puncture catheter 1, the pitch of the coil 23 may increase toward the distal side or toward the proximal side.

In a state where no external force is applied to the puncture catheter 1, the pitch of the coil 23 may be 2. 0 times or less, 1.8 times or less, or 1.5 times or less of the wire diameter of the wire 21. In a state where no external force is applied to the puncture catheter 1, the pitch of the coil 23 can be 1.0 times or more of the wire diameter of the wire 21. In a state where no external force may be applied to the puncture catheter 1, the pitch of the coil 23 is the same as the wire diameter of the wire 21. With the above configuration, the inner surface 10c of the resin tube 10 can be easily protected by the tubular member 20.

The holes 24 formed in the pipe 26 may be bottomed holes or through holes.

The outer shape of each of the holes 24 of the pipe 26 when observed in the radial direction of the pipe 26 may be a polygonal shape such as a triangular shape, a quadrangular shape, or a pentagonal shape, a circular shape, an elliptical shape, or a combination thereof. Note that the polygonal shape includes a rounded-corner polygonal shape in which corners are rounded and a polygonal shape in which at least a portion of a side is curved, in addition to a polygonal shape in which vertices of corners are clear and in which sides are straight lines.

The depth direction of each of the holes 24 may be inclined with respect to the radial direction y of the resin tube 10. For example, as shown in FIG. 8, one hole 24 may have a first opening 241 located on the inner surface 20c side of the tubular member 20 and a second opening 242 located on the outer surface 20d side of the tubular member 20, a distal end 241a of the first opening 241 may be located distal to a distal end 242a of the second opening 242, and a proximal end 241b of the first opening 241 may be located distal to a proximal end 242b of the second opening 242. This makes it easy to suppress the puncture needle 30 from being caught in the hole 24.

The grooves 25 formed in the pipe 26 may be bottomed grooves or through grooves.

The grooves 25 may extend in a direction perpendicular to the longitudinal direction of the pipe 26. The grooves 25 may extend in the longitudinal direction of the pipe 26. The grooves 25 may extend so as to be inclined with respect to the longitudinal direction of the pipe 26 and to the direction perpendicular to the longitudinal direction of the pipe 26.

The holes 24 and the grooves 25 may be formed in the pipe 26.

In the longitudinal direction x of the resin tube 10, the length of the tubular member 20 may be shorter than the length of the resin tube 10.

The length of the tubular member 20 in the longitudinal direction x of the resin tube 10 can be 20 mm or more, 70 mm or more, 120 mm or more, or the like. The length of the tubular member 20 in the longitudinal direction x of the resin tube 10 can be 200 mm less, 250 mm or less, 300 mm or less, or the like. The length of the tubular member 20 in the longitudinal direction x of the resin tube 10 refers to the longest length among lengths of the tubular member 20 in the longitudinal direction x of the resin tube 10. For use as a puncture catheter for puncturing the myocardium, the length of the tubular member 20 may be 70 mm in the longitudinal direction x of the resin tube 10.

The outer diameter of the tubular member 20 can be 0.5 mm or more, 1.0 mm or more, 1.5 mm or more, or the like. The outer diameter of the tubular member 20 can be 2.0 mm or less, 2.5 mm or less, 3.0 mm or less, or the like. When the outer shape of the tubular member 20 is not a perfect circle when observed in a direction perpendicular to the radial direction of the tubular member 20, the diameter of a circle circumscribing the tubular member 20 is defined as the outer diameter of the tubular member 20. For use as a puncture catheter for puncturing the myocardium, the outer diameter of the tubular member 20 may be 1.3 mm.

The wire 21 may have a constant wire diameter. The wire 21 may have a different wire diameter for each portion.

The wire diameter of the wire 21 can be 0.1 mm or more, 0.2 mm or more, 0.3 mm or more, or the like. The wire diameter of the wire 21 can be 0.5 mm or less, 0. 6 mm or less, 0.7 mm or less, or the like. In the case where the outer shape of the wire 21 when observed in the axial direction of the wire 21 is not a perfect circle, the diameter of a circle circumscribing the wire 21 is defined as the wire diameter of the wire 21. When the puncture catheter 1 is used to puncture the myocardium, the outer shape of the wire 21, when observed in the axial direction of the wire 21, may be a rectangular shape, that the length of each short side of the rectangular shape is 0.2 mm and the length of each long side thereof is 0.3 mm, and that the wire diameter of the wire 21 is 10.13.

The puncture catheter 1 may include only one or a plurality of the tubular members 20.

The puncture needle 30 may be used to puncture an organ in the body.

The puncture needle 30 can be made of, for example, a metal or a resin. The puncture needle 30 may be entirely made of only a metal or may be entirely made of only a resin. A portion of the puncture needle 30 may be made of a metal while the other portion is made of a resin.

The puncture needle 30 may be made of only a metal. An example of the metal constituting the puncture needle 30 is, for example, stainless steel such as SUS304 or SUS316, platinum, nickel, cobalt, chromium, titanium, tungsten, gold, a Ni—Ti alloy, a Co—Cr alloy, or a combination thereof.

An example of the resin constituting the puncture needle 30 is, for example, polyether ether ketone (PEEK), polycarbonate (PC), or the like. When the puncture needle 30 is made of only a resin without using any metal, the puncture catheter 1 can be used even for a patient with metal allergy.

The length of the puncture needle 30 in the longitudinal direction x of the resin tube 10 can be 2 mm or more, 3 mm or more, or 4 mm or more. The length of the puncture needle 30 in the longitudinal direction x of the resin tube 10 can be 50 mm H less, 30 mm or less, 10 mm or less, or the like. The length of the puncture needle 30 in the longitudinal direction x of the resin tube 10 refers to the longest length among lengths of the puncture needle 30 in the longitudinal direction x of the resin tube 10. For use as a puncture catheter for puncturing the myocardium, the length of the puncture needle 30 in the longitudinal direction x of the resin tube 10 may be 5 mm.

The length of the puncture needle 30 in the radial direction y of the resin tube 10 can be 0.2 mm or more, 0.3 mm or more, 0.4 mm or more, or the like. The length of the puncture needle 30 in the radial direction y of the resin tube 10 can be 10 mm or less, 5 mm or less, 1 mm or less, or the like. The length of the puncture needle 30 in the radial direction y of the resin tube 10 refers to the longest length among lengths of the puncture needle 30 in the radial direction y of the resin tube 10. For use as a puncture catheter for puncturing the myocardium, the length of the puncture needle 30 may be 0.45 mm in the radial direction y of the resin tube 10.

The puncture needle 30 may have a lumen extending in the longitudinal direction x of the resin tube 10 and a hole 31 through which the lumen is in communication with the outside of the puncture needle 30. A liquid such as a cell preparation or a drug solution carried through the lumen of the puncture needle 30 is carried to a target tissue through the hole 31.

The hole 31 can allow a liquid to be discharged from the lumen of the puncture needle 30 outwardly in the radial direction of the resin tube 10. Therefore, the hole 31 may be located proximal to the distal end of the puncture needle 30 and distal to the proximal end of the puncture needle 30.

The puncture needle 30 may be provided with only one or a plurality of the holes 31. When only one hole 31 is formed in the puncture needle 30, it is easy to administer a liquid such as a cell preparation or a drug solution in a pinpoint manner. When a plurality of the holes 31 are formed in the puncture needle 30, it is easy to administer a liquid such as a cell preparation or a drug solution in a wide range.

The outer shape of each hole 31 of the puncture needle 30 when observed in a direction perpendicular to the longitudinal direction x of the resin tube 10 can be a polygonal shape such as a triangular shape, a quadrangular shape, or a pentagonal shape, a circular shape, an elliptical shape, or a combination thereof.

The puncture catheter 1 may include a handle 40 that controls the curving direction of the resin tube 10. The handle 40 is a part to be held by a user and may have a shape that allows the user to easily hold the handle 40. The handle 40 may be connected to the proximal end portion of the resin tube 10.

Control of the curving direction of the resin tube 10 by operation of the handle 40 is enabled by, for example, providing a wire in the puncture catheter 1. More specifically, the distal end portion of the wire is fixed to the distal end portion of the resin tube 10, the proximal end portion of the wire is fixed to the handle 40, and the wire is moved toward the proximal side by operating the handle 40, whereby the resin tube 10 can be curved.

The constituent material of the handle 40 is not particularly limited. For example, a synthetic resin such as a polyolefin resin such as polypropylene (PP) or polyethylene (PE), a polyester resin such as polyethylene terephthalate (PET), a polycarbonate resin, an ABS resin, or a polyurethane resin can be used.

As shown in FIG. 3, the resin tube 10 may have a curving section 11 which is curved by operating the handle 40, and a non-curving section 12 which is located proximal to the curving section 11 and is not curved by operating the handle 40. In this case, the tubular member 20 may be disposed in the curving section 11. With the above configuration, in a curved portion of the resin tube 10, the puncture needle 30 is less likely to stick into the resin tube 10 and the puncture needle 30 is less likely to pass through the resin tube 10.

The tubular member 20 may be disposed in the non-curving section 12. However, the tubular member 20 need not be disposed in the non-curving section 12.

As shown in FIGS. 1 to 7, at least one electrode 50 may be disposed on the outer surface 10d of the resin tube 10. The number of the electrodes 50 disposed on the outer surface 10d of the resin tube 10 may be one or two but may be three or more. The number of the electrodes 50 disposed on the outer surface 10d of the resin tube 10 can be, for example, ten or less, eight or less, or the like.

As each electrode 50, it is preferable to use an electrode capable of capturing an electrical signal of an organ or an electrode capable of capturing a flowing current. The electrical signal includes information on a current, a voltage, a potential, and the like. For example, an electrode capable of capturing the intensity of a current, the direction in which the current flows, and the like is preferable.

Each electrode 50 may be annular or cylindrical.

Each electrode 50 can be made of, for example, a conductive material such as platinum or stainless steel, but may contain a radiopaque material such as platinum so that the position of the electrode can be easily recognized under X-ray fluoroscopy. Materials constituting respective electrodes 50 may be all the same or may be different from each other.

Each electrode 50 may be disposed on the outer surface 10d of the distal portion of the resin tube 10 and may be disposed on the outer surface 10d of the distal end portion of the resin tube 10.

The tubular member 20 may be disposed distal to the proximal end of the electrode 50 located most proximally. The entire tubular member 20 may be disposed distal to the proximal end of the electrode 50 located most proximally. The tubular member 20 is not necessarily disposed proximal to the proximal end of the electrode 50 located most proximally. Only a portion of the tubular member 20 may be disposed distal to the proximal end of the electrode 50 located most proximally while the remaining portion of the tubular member 20 is disposed proximal to the proximal end of the electrode 50 located most proximally.

The tubular member 20 may be disposed proximal to the distal end of the electrode 50 located most distally. The entirety of the tubular member 20 may be disposed proximal to the distal end of the electrode 50 located most distally. The tubular member 20 need not be disposed distal to the distal end of the electrode 50 located most distally. Note that only a portion of the tubular member 20 may be disposed proximal to the distal end of the electrode 50 located most distally while the remaining portion of the tubular member 20 is disposed distal to the distal end of the electrode 50 located most distally.

As shown in FIG. 2, the puncture catheter 1 may include a tube 60 connected to the proximal end portion of the puncture needle 30 and having a lumen extending in the longitudinal direction x of the resin tube 10. The proximal end portion of the puncture needle 30 and the distal end portion of the tube 60 may be connected to each other so that the lumen of the puncture needle 30 and the lumen of the tube 60 are in communication with each other.

The tube 60 may be movable in the longitudinal direction x of the resin tube 10 in the lumen 10e of the resin tube 10 and the lumen 20e of the tubular member 20.

The tube 60 can be formed of, for example, a material exemplified as a material capable of forming the puncture needle 30.

The tube 60 may be constituted by only one tubular member or may be constituted by a plurality of tubular members.

The present application claims the benefit of priority based on Japanese Patent Application No. 2023-215765, filed on Dec. 21, 2023. The entire content of the description of Japanese Patent Application No. 2023-215765, filed on Dec. 21, 2023, is incorporated herein by reference.

### REFERENCE SIGNS LIST

1 puncture catheter
10 resin tube
10a distal end
10b proximal end
10c inner surface
10d outer surface
10e lumen
11 curving section
12 non-curving section
20 tubular member
20a distal end
20b proximal end
20c inner surface
20d outer surface
20e lumen
21 wire
22 braid
23 coil
24 hole
241 first opening
241a distal end
241b proximal end
242 second opening
242a distal end
242b proximal end
25 groove
26 pipe
30 puncture needle
31 hole
40 handle
50 electrode
60 tube

Although the disclosure has been described with respect to only a limited number of embodiments, those skilled in the art, having benefit of this disclosure, will appreciate that various other embodiments may be devised without departing from the scope of the present disclosure.

Accordingly, the scope of one or more embodiments of the invention should be limited only by the attached claims.

1. A puncture catheter comprising:
a resin tube that has a longitudinal direction and that has a lumen extending in the longitudinal direction;
a tubular member that has an outer surface in contact with an inner surface of a distal portion of the resin tube; and
a puncture needle that is for medical use and that is movable in the longitudinal direction in the lumen of the resin tube and a lumen of the tubular member,
wherein the tubular member is formed of a braid in which a wire is braided, a coil around which a wire is wound, a pipe in which a plurality of holes or grooves are formed, or a combination thereof.

2. The puncture catheter according to claim 1,
wherein a gap is present in the lumen of the tubular member, and
wherein an inner surface of the tubular member is exposed to the gap.

3. The puncture catheter according to claim 1,
wherein:
the puncture catheter comprises a handle that controls a curving direction of the resin tube,
the resin tube has a curving section that is curved by operating the handle and a non-curving section that is located proximal to the curving section and that is not curved by operating the handle, and
the tubular member is disposed in the curving section of the resin tube.

4. The puncture catheter according to claim 3, wherein the tubular member is not disposed in the non-curving section of the resin tube.

5. The puncture catheter according to claim 1,
wherein:
three or more electrodes are disposed on an outer surface of the resin tube,
the tubular member is disposed distal to a proximal end of an electrode of the three or more electrodes located most proximally, and
the tubular member is not disposed proximal to the proximal end of the electrode located most proximally.

6. The puncture catheter according to claim 1, wherein the tubular member is made of a metal.

7. The puncture catheter according to claim 1, wherein an inner surface of the tubular member and the puncture needle are contactable with each other.

8. The puncture catheter according to claim 2,
wherein:
the puncture catheter comprises a handle that controls a curving direction of the resin tube,
the resin tube has a curving section that is curved by operating the handle and a non-curving section that is located proximal to the curving section and that is not curved by operating the handle, and
the tubular member is disposed in the curving section of the resin tube.

9. The puncture catheter according to claim 8, wherein the tubular member is not disposed in the non-curving section of the resin tube.

10. The puncture catheter according to claim 2,
wherein:
three or more electrodes are disposed on an outer surface of the resin tube,
the tubular member is disposed distal to a proximal end of an electrode of the three or more electrodes located most proximally, and
the tubular member is not disposed proximal to the proximal end of the electrode located most proximally.

11. The puncture catheter according to claim 2, wherein the tubular member is made of a metal.

12. The puncture catheter according to claim 2, wherein an inner surface of the tubular member and the puncture needle are contactable with each other.
